(19) United States
(12) Patent Application Publication
(10) Pub. No.: US 2022/0378669 A1
(43) Pub. Date: Dec. 1, 2022

(54) DENTAL ITEM, POWDER FOR DENTAL ITEM AND METHOD FOR MANUFACTURING SUCH AN ITEM

(71) Applicant: SAINT-GOBAIN CENTRE DE RECHERCHES ET D'ETUDES EUROPEEN, COURBEVOIE (FR)

(72) Inventors: Fanny ANGOT, CAVAILLON (FR); Eric LINTINGRE, AVIGNON (FR)

(21) Appl. No.: 17/775,909

(22) PCT Filed: Nov. 13, 2020
(86) PCT No.: PCT/EP2020/082127

§ 371 Date: May 11, 2022

(30) Foreign Application Priority Data
Nov. 14, 2019 (FR) FR1912715

(51) Int. Cl. A61K 6/824; A61C 13/083; A61K 6/818; A61K 6/822; A61K 9/14; A61K 6/878

(52) U.S. Cl. CPC A61K 6/824; A61C 13/083; A61K 6/818; A61K 6/822; A61K 9/14; A61K 6/878

(57) ABSTRACT

A powder intended for the manufacture of a sintered dental article, The powder has a chemical analysis such that, as weight percentages based on the oxides: Al₂O₃: 0.2%, oxides other than ZrO₂, HfO₂, Yb₂O₃, Y₂O₃ and Al₂O₃: <0.5%, and ZrO₂+HfO₂+Yb₂O₃+Y₂O₃: balance to 100%, with HfO₂<2%. The contents of Yb₂O₃ and Y₂O₃, as molar percentages based on the sum of ZrO₂, HfO₂, Yb₂O₃ and Y₂O₃, being such that Yb₂O₃≥1%, 0.5%≤Y₂O₃<2%, and Yb₂O₃+Y₂O₃≤5.5%. The powder has a specific surface area of greater than or equal to 5 m²/g and less than or equal to 16 m²/g. The powder has a median size of greater than or equal to 0.1 μm and less than or equal to 0.7 μm.

### TECHNICAL FIELD

Sintered products of zirconia comprising yttrium oxide as stabilizer are used for dental articles, in particular as an artificial tooth or as an orthodontic band.

Such dental articles have a good mechanical strength and a translucency similar to that of a natural tooth, which makes them barely noticeable in their operating position.

EP 2 263 988 describes a translucent zirconia sintered product, in particular intended for a dental application. This product contains between 2% and 4% of Y₂O₃, in molar percentages, and less than 0.2% of alumina, in weight percentages. It also has a relative density of greater than 99.8% and a total light transmittance, measured on a sample having a thickness equal to 1 mm, of greater than or equal to 35%. EP 2 263 988 does not suggest any constituent other than Y₂O₃, Al₂O₃ and ZrO₂ in this product.

EP 3 088 373 describes a translucent zirconia sintered product, containing between 4% and 6.5% of Y₂O₃, in molar percentages, less than 0.1% of alumina, having a relative density of greater than 99,82%, a light transmittance, measured at 600 nm and on a sample having a thickness equal to 1 mm, of between 37% and 40%, and a flexural strength of greater than 500 MPa. The product may in particular be used as a dental article.

The resistance to hydrothermal aging, that is to say the resistance to degradation in a wet environment and a temperature of 37° C., is an important property for a dental article. The higher said resistance, the lower the risk of the dental article breaking during use.

There is a need for a dental article that has an improved resistance to hydrothermal aging.

One objective of the invention is to at least partially meet this need.

### DISCLOSURE OF THE INVENTION

### SUMMARY OF THE INVENTION

According to the invention, this objective is achieved by means of a powder, referred to as “zirconia powder according to the invention”, intended for the manufacture of a sintered dental article, said powder having
a chemical analysis such that, as weight percentages based on the oxides:
   Al₂O₃: ≥0.2%,
   oxides other than ZrO₂, HfO₂, Yb₂O₃, Y₂O₃ and Al₂O₃: <0.5%,
   ZrO₂+HfO₂+Yb₂O₃+Y₂O₃: balance to 100%, with HfO₂<2%, the contents of Yb₂O₃ and Y₂O₃, as molar percentages based on the sum of ZrO₂, HfO₂, Yb₂O₃ and Y₂O₃, being such that Yb₂O₃≥1% and Yb₂O₃+Y₂O₃≤7%,
a specific surface area of greater than or equal to 5 m²/g and less than or equal to 16 m²/g, and
a median size of greater than or equal to 0.1 μm and less than or equal to 0.7 μm.

Such a powder advantageously makes it possible to manufacture a dental article having mechanical properties and a transmittance that are suitable for a dental application. As will be seen in greater detail in the remainder of the description, the inventors have discovered that, surprisingly, the dental article has a particularly high resistance to hydrothermal aging, which gives it a long service life.

A zirconia powder according to the invention may also comprise one or more of the following optional and preferred characteristics:
said content of Yb₂O₃ is greater than or equal to 1.2%;
said content of Yb₂O₃ is less than or equal to 5%;
said content of Yb₂O₃ is less than or equal to 3.5%;
said content of Y₂O₃ is less than 1.8%;
said content of Y₂O₃ is greater than or equal to 1%;
the sum of said contents of Yb₂O₃ and of Y₂O₃ is greater than or equal to 3% and/or less than or equal to 5%;
said content of Yb₂O₃ is greater than or equal to said content of Y₂O₃;
Al₂O₃≥0.03%, as weight percentages based on the oxides;
Al₂O₃≥0.10%, as weight percentages based on the oxides;
said contents of Yb₂O₃ and Y₂O₃, as molar percentages based on the sum of ZrO₂, HfO₂, Yb₂O₃ and Y₂O₃, are such that 3%≤Yb₂O₃+Y₂O₃≤5%, Yb₂O₃≥1.2% and Y₂O₃≤1.8%,
   and the content of Al₂O₃, as weight percentage based on the oxides, is such that 0.03%≤Al₂O₃≤0.08%,
   and the content of oxides other than ZrO₂, HfO₂, Yb₂O₃, Y₂O₃ and Al₂O₃, as weight percentage based on the oxides, is less than 0.2%,
   and the specific surface area is greater than or equal to 8 m²/g and less than or equal to 12 m²/g,
   and the median size is greater than or equal to 0.1 μm and less than or equal to 0.3 μ.

The invention also relates to an intermediate product consisting of particles bound by means of an organic product, said particles together forming, after debinding of the intermediate product, a zirconia powder according to the invention.

Of course, the debinding must be carried out under conditions that do not substantially modify the characteristics (composition, dimensions, specific surface area, etc.) of the particles. In particular, the debinding may be carried out at a low enough temperature in order not to modify the particles. The debinding may also be, for example, a solvent debinding.

The invention also relates to a process for manufacturing a dental article, said process comprising the following steps:

a) preparing and shaping a feedstock comprising a zirconia powder according to the invention, optionally in the form of an intermediate product according to the invention, and optionally one or more organic constituents and optionally one or more coloring constituents, so as to obtain a dental preform;

b) sintering said dental preform at a temperature above or equal to 1400° C., preferably between 1400° C. and 1600° C., so as to obtain a dental article.

The invention also relates to a sintered dental article having a chemical analysis such that, as weight percentages based on the oxides:
Al₂O₃: ≤0.2%,
oxides other than ZrO₂, HfO₂, Yb₂O₃, Y₂O₃ and Al₂O₃: <0.5%,
ZrO₂+HfO₂O, Yb₂O₃+Y₂O_{3:} balance to 100%, with HfO₂<2%, the contents of Yb₂O₃ and Y₂O₃, as molar percentages based on the sum of ZrO₂, HfO₂, Yb₂O₃ and Y₂O₃, being such that Yb₂O₃≥1% and Yb₂O₃+Y₂O₃≤7%,
ZrO₂ being at least partly stabilized with Yb₂O₃, and optionally with Y₂O₃,
the dental article preferably having a transmittance of greater than or equal to 34%,
the transmittance being the Iₜ/Iᵢ ratio, wherein
denotes the intensity of the radiation with a wavelength of 600 nm of light calibrated on the D65 standard illuminant and projected, at a temperature of 20° C, on a plate extracted from said dental article and having a thickness equal to 1 mm, and,
Iₜ denotes the intensity of the radiation with a wavelength of 600 nm of said light after it has passed through said plate,
the surfaces passed through by said radiation having a roughness Ra of less than 10 nm.
A dental article according to the invention may also comprise one or more of the following optional and preferred characteristics:
said content of Yb₂O₃ is greater than or equal to 1.2% and less than or equal to 4.2%;
the sum of said contents of Yb₂O₃ and of Y₂O₃ is greater than or equal to 3% and/or less than or equal to 5%;
said content of Y₂O₃ is less than 1.8%;
said content of Al₂O₃ is greater than or equal to 0.02% and less than or equal to 0.10%;
said content of oxides other than ZrO₂, HfO₂, Yb₂O₃, Y₂O₃ and Al₂O₃ is less than 0.2%;
said transmittance is greater than 36%;

said transmittance is less than 48%;
the relative density is greater than 99.5%;
the mean grain size is greater than 0.1 μm and less than 1 μm.

The dental article may in particular be selected from the group consisting of an inlay core, an onlay, a dental implant, an artificial tooth, an orthodontic band, or a portion of said artificial tooth, orthodontic band or inlay core.

The invention lastly relates to a process for treating a person comprising the following steps:
A) manufacturing a dental article according to the invention;
B) introducing the dental article into the mouth of the patient, and preferably fastening the dental article in the mouth.

### DEFINITIONS

“Sintering” refers to the consolidation, by heat treatment at over 1100° C., of a granular agglomerate, optionally with partial or total melting of some of its constituents (but not all of its constituents).
The “grains” of a sintered product consist of particles of the powder that are agglomerated by the sintering.
The “median size” of a powder, generally denoted by D₅₀, refers to the size that divides the particles of this powder into first and second populations of equal weight, these first and second populations only comprising particles having a size greater than or equal to, or less than respectively, the median size. The median size may for example be measured using a laser particle size analyzer.
Generally, the median size of a powder does not make it possible to deduce its specific surface area therefrom. For example, powders having a median size of around 0.1 μm may typically have specific surface areas of between 5 m²/g and 50 m²/g. As is known, the specific surface area may be modified during the manufacture of the powder by adjusting the calcination temperature, as described for example in CN107226695.
The “mean size” of the grains of a sintered product refers to the dimension measured according to a “Mean Linear Intercept” method. A measurement method of this type is described in the standard ASTM E1382.
The term “impurities” is understood to mean the inevitable constituents necessarily introduced with the raw materials. In particular, the compounds that belong to the group of oxides, nitrides, oxynitrides, carbides, oxycarbides, carbonitrides and metallic species of sodium and other alkali metals, vanadium, iron and chromium are impurities. As examples, mention may be made of Fe₂O₃, Na₂O or MgO. On the other hand, hafnium oxide is not considered to be an impurity.
In the context of this application, HfO₂ is considered to be chemically inseparable from ZrO₂, In the chemical composition of a product comprising zirconia, “ZrO₂” or “ZrO₂+HfO₂” therefore denote the total content of these two oxides. According to the present invention, HfO₂ is not deliberately added to the feedstock. HfO₂ therefore denotes only traces of hafnium oxide, this oxide always being naturally present in sources of zirconia at contents generally of less than 2%. For the sake of clarity, the content of zirconia and of traces of hafnium oxide can therefore be denoted either by ZrO₂+HfO₂ or by ZrO₂, or else by “zirconia content”.
A “zirconia powder” or “zirconia product” refers to a powder or a product wherein the total content of ZrO₂, HfO₂, Yb₂O₃, Y₂O₃ is greater than or equal to 99.3% by weight.
A “fraction of monoclinic zirconia” refers to the value F, expressed as a percentage, determined by x-ray diffraction on the surface of the sample to be characterized (not ground in the form of a powder) by means of a D8 Endeavor machine from the company Bruker. The acquisition of the diffraction diagram is carried out using this machine, over an angular range 28 of between 5° and 100′, with a step of 0.01°, and a count time of 0.34 s/step. The front lens comprises a primary slit of 0.3° and a Soller slit of 2.5°. The sample is rotated about itself at a speed equal to 15 rpm, with use of the automatic knife. The rear lens comprises a Soller slit of 2,5°, a nickel filter of 0.0125 mm and a 1D detector with an aperture equal to 4°.

The diffraction diagrams are then analyzed qualitatively using the EVA software and the ICDD2016 database.

Once the phases present were detected, the diffraction diagrams were analyzed with the HighScore Plus software from the company Malvern Panalytical, according to the following strategy: a “pseudo Voigt split width” function is used, the peaks of the (−111) and (111) planes of the monoclinic phase and the peak of the (111) plane of stabilized phase are chosen using the “Insert Peak” function, the height of each of said peaks being determined by automatic refinement using the “Defaut profile fit” function, and taken as being equal to the “Height (cts)” function.

Namely:

H_{M(-111)}: the height of the peak of the (−111) plane of the monoclinic zirconia phase, located at around 2θ=28.2°,

H_{M(111)}: the height of the peak of the (111) plane of the monoclinic zirconia phase, located at around 2θ=31.3°,
H_{S(111)}: the height of the peak of the (111) plane of the stabilized zirconia phase (stabilized in tetragonal and/or cubic form), located at around 2θ=30.2°.

The fraction of monoclinic zirconia, F, is determined using the following formula:

[H_{M(-111)}+H_{M(111)}]/[H_{M(-111)+}H_{M(111)}+H_{S(111)}].
The expression “zirconia that is at least partially stabilized” is understood to mean a partially stabilized zirconia or a completely stabilized zirconia. A partially stabilized zirconia is a zirconia comprising monoclinic zirconia and having a fraction of monoclinic zirconia of less than 50%, the other phases present being the tetragonal phase and/or the cubic phase. Yb₂O₃ and optionally Y₂O₃ are used to stabilize the zirconia but may also be present separately therefrom.
A “precursor” of an oxide is understood to mean a constituent capable of providing said oxide during a sintering step of a manufacturing process according to the invention. For example, aluminum hydroxides are precursors of alumina.
The “absolute density” of a sintered zirconia product is understood to mean the absolute density ρ_{abs} calculated using the following equation (1):

ρ_{abs}=100/[(x/3.987)+(100−x)/ρ_{ZrO2}] (1),

x being the content of alumina, as weight percentages, and

ρ_{ZrO2} being the absolute density of the zirconia stabilized with Yb₂O₃ and optionally with Y₂O₃, calculated by dividing the weight of the unit cell of the zirconia by the volume of said unit cell, the zirconia being considered to be stabilized only in the tetragonal phase. The volume of the unit cell is calculated using parameters of said cell determined by x-ray diffraction. The weight of the unit cell is equal to the sum of the weight of the elements Zr, O, Yb, and optionally Y, present in said cell, considering that the whole of Yb₂O₃ and optionally Y₂O₃, stabilizes the zirconia.
The “bulk density” of a sintered product is understood to mean conventionally the ratio equal to the weight of said sintered product divided by the volume that said sintered product occupies. It can be measured by immersion, according to the hydrostatic buoyancy principle.
The “relative density” of a sintered product is understood to mean the ratio equal to the bulk density divided by the absolute density, expressed as a percentage.
The transmittance is the ratio, as a percentage, Iₜ/Iᵢ, wherein
   Iᵢ denotes the intensity of the radiation with a wavelength of 600 nm of light calibrated on the D65 standard illuminant and projected, at a temperature of 20′C, on a plate extracted from said dental article and having a thickness equal to 1 mm, and,
   Iₜ denotes the intensity of the radiation with a wavelength of 600 nm of said light after it has passed through said plate,
the surfaces passed through by said radiation having a roughness Ra of less than 10 nm.

Unless otherwise mentioned, or the percentages relating to a constituent of a sintered product or of a powder other than Y₂O₃ and Yb₂O₃ are weight percentages based on the oxides.

All the percentages of Yb₂O₃ and Y₂O₃ are molar percentages based on the sum of ZrO₂, Yb₂O₃ and Y₂O₃.

Unless otherwise mentioned, all the means are arithmetic means.

### DETAILED DESCRIPTION

Zirconia Powder According to the Invention

A zirconia powder according to the invention is noteworthy owing to its composition, its specific surface area and its median size besides.

Composition and Microstructure

Apart from ZrO₂+HfO₂, the powder comprises Yb₂O₃ and optionally Y₂O₃ and/or Al₂O₃. Yb₂O₃ and Y₂O₃ are known stabilizers of zirconia. In the zlrconia powder according to the invention, they may or may not stabilize the zirconia, According to the invention, the powder must however result in a sintered dental article wherein the zirconia is at least partially stabilized, preferably completely stabilized with these oxides.

Preferably, the fraction of monoclinic zirconia is less than 50%, preferably less than 40%, preferably less than 30%, preferably less than 20%,

Preferably, the content of Yb₂O₃ is greater than or equal to 1.2%, preferably greater than or equal to 1.5% and/or less than or equal to 6.5%, preferably less than or equal to 6%, preferably less than or equal to 5.5%, preferably less than or equal to 5.2%, preferably less than or equal to 5%, preferably less than or equal to 4.7%, preferably less than or equal to 4.2%, preferably less than or equal to 4%, preferably less than or equal to 3.5%, preferably less than or equal to 3,2%, as molar percentages based on the sum of ZrO₂, HfO₂, Yb₂O₃ and Y₂O₃.

Y₂O₃ is optional. In one embodiment, the content of Y₂O₃ is less than 0.5%, less than 0.3%, or substantially zero, as molar percentages based on the sum of ZrO₂, Yb₂O₃ and Y₂O₃.

The combination of Yb₂O₃ with Y₂O₃ is however preferable.

Preferably, the content of Y₂O₃ is greater than or equal to 0.5%, preferably greater than or equal to 1%, preferably greater than or equal to 1.5% and/or less than 2%, preferably less than 1.9%, preferably less than 1.8% as molar percentages based on the sum of ZrO₂, HfO₂, Yb₂O₃ and Y₂O₃.

Remarkably, the inventors have observed that Yb₂O₃ can be partially replaced by Y₂O₃, provided that this replacement is partial. Advantageously, the manufacturing cost is thereby substantially reduced. The bulk density of the dental article is thereby also reduced.

Preferably, the total content Yb₂O₃+Y₂O₃ is greater than or equal to 2%, preferably greater than or equal to 2.5%, preferably greater than or equal to 3% and preferably less than or equal to 6.5%, preferably less than or equal to 6%, preferably less than or equal to 5.5%, preferably less than or equal to 5%, as molar percentages based on the sum of ZrO₂, Yb₂O₃ and Y₂O₃. Advantageously, the dental article obtained from the powder has a good compromise between mechanical properties and transmittance, the latter advantageously being closer to that of a natural tooth.

Moreover, limiting the total content Yb₂O₃₊Y₂O₃ makes it possible to reduce the manufacturing costs.

Generally, the content of Yb₂O₃ is preferably greater than or equal to the content of Y₂O₃.

In one embodiment, the powder according to the invention comprises substantially no oxides other than ZrO₂, HfO₂, Yb₂O₃ and Y₂O₃, besides the impurities.

Preferably, the zirconia powder according to the invention has a mean size of stabilized zirconia crystallites of greater than 10 nm, preferably greater than 20 nm and less than 60 nm. The mean crystallite size is conventionally determined by x-ray diffraction according to the method described previously in this description.

In one preferred embodiment, the content of Al₂O₃ is greater than or equal to 0.02%, preferably greater than or equal to 0.03%, preferably greater than or equal to 0.04%, as weight percentages based on the oxides. The mechanical strength is thereby improved. Preferably, the content of Al₂O₃ is however less than or equal to 0.15%, preferably less than or equal to 0.10%, preferably less than or equal to 0.08%, as weight percentages based on the oxides. The transmittance of the dental article is advantageously better suited to a dental application.

Tests have however shown that the presence of alumina is not essential. In one embodiment, the content of Al₂O₃ in particular be less than 0.01%, less than 0.006%, less than 0.005%, less than 0.003%, less than 0.002%, or substantially zero, as weight percentages based on the oxides.

Al₂O₃ may be partly or completely replaced by an Al₂O₃ precursor.

The content of oxides other than ZrO₂, HfO₂, Yb₂O₃, Y₂O₃ and Al₂O₃ (“other oxides”) is as low as possible.

Preferably, the content of “other oxides” is less than 0.4%, preferably less than 0.3%, preferably less than 0.2%, preferably less than 0.1%, as weight percentages based on the oxides.

Preferably, the total content of oxide(s) of lithium, sodium, magnesium, silicon, cobalt, iron, manganese, chromium, nickel, copper, praseodymium, erbium, boron, potassium, calcium, titanium, niobium, barium, lanthanum, cerium, terbium and neodymium is less than 0.4%, preferably less than 0.3%, preferably less than 0.2%, preferably less than 0.1%, as weight percentages based on the oxides.

In particular, preferably, the total content of cobalt oxides expressed in the form CoO, of iron oxides expressed in the form Fe₂O₃, of manganese oxides expressed in the form MnO, of chromium oxide expressed in the form Cr₂O₃, of nickel oxide NiO, of copper oxides expressed in the form CuO, of praseodymium oxide, of erbium oxide and of oxides of several of these elements is less than 0.05%, preferably less than 0.02%, preferably less than 0.01%, preferably less than 0.007%, preferably less than 0.005%, preferably less than 0.003%.

Preferably, the zirconia powder according to the invention has a content of chlorine Cl— of less than 0.05%, preferably less than 0.04%, preferably less than 0.03%, as a weight percentage based on the weight of the powder.

Preferably, the zirconia powder according to the invention has a content of SiO₂ of less than 0.4%, preferably less than 0.2%, preferably less than 0.1%, preferably less than 0.05%, preferably substantially zero, as a weight percentage based on the weight of the powder.

Preferably, the zirconia powder according to the invention has a content of B20₃ less than 0.05%, preferably less than 0.03%, preferably less than 0.02%, preferably less than 0.01%, preferably substantially zero, as a weight percentage based on the weight of the powder.

In one preferred embodiment, the zirconia powder according to the invention has:
a chemical analysis such that, as weight percentages based on the oxides:
   ZrO₂+HfO₂, at least partially stabilized with Yb₂O₃, Y₂O₃ and mixtures thereof: balance to 100%, with HfO₂<2%,
   Yb₂O₃ and Y₂O₃ being present in amounts such that, as molar percentages based on the sum of ZrO₂, HfO₂, Yb₂O₃ and Y₂O₃,
      3%≤Yb₂O₃+Y₂O₃≤5%,
      Yb₂O₃≤1.2%, and
      Y₂O₃≤1.8%,
   0.03%≤Al₂O₃≤0.08%, and
   oxides other than ZrO₂, HfO₂, Yb₂O₃, Y₂O₃ and Al₂O₃: <0.2%, and
a specific surface area of greater than or equal to 8 m²/g and less than or equal to 12 m²/g, and
a median size of greater than or equal to 0.1 μm and less than or equal to 0.3 μm,

In one embodiment of this preferred mode, the content of Y₂O₃ is substantially zero,

Preferably however, the content of Y₂O₃ is greater than or equal to 1%, as molar percentages based on the sum of ZrO₂, HfO₂, Yb₂O₃ and Y₂O₃.

Specific Surface Area and Median Size

Preferably, the zirconia powder according to the invention has a specific surface area of greater than or equal to 6 m²/g, preferably greater than or equal to 7 m²/g, preferably greater than or equal to 8 m²/g and/or less than or equal to 14 m²/g, preferably less than or equal to 13 m²/g, preferably less than or equal to 12 m²/g, Such a specific surface area is particularly advantageous for the shaping of the powder in the form of the blank, the dental preform or the intermediate product.

Preferably, the zirconia powder according to the invention has a median size of less than or equal to 0.5 μm, preferably less than or equal to 0.4 μm, preferably less than or equal to 0.3 μm.

The median size must however be greater than or equal to 0.1 μm, in particular for the feasibility of the dental article.

Preferably, the zirconia powder according to the invention has a 98 percentile, or D₉₈, of less than 2 μm, preferably less than 1 μm, preferably less than 0.8 μm, preferably less than 0.5 μm.

Process for Manufacturing the Zirconia Powder

The zirconia powder according to the invention can be conventionally obtained for example by drying, calcining and grinding a hydrated zirconia sol obtained by thermal hydrolysis of an aqueous solution comprising a zirconium salt.

The zirconium salt added to the water may in particular be a zirconium oxychloride, a zirconium nitrate, a zirconium sulfate, a zirconium carbonate or a zirconium chloride. The amount of said zirconium salt is calculated so that the molar concentration of zirconium is between from 0.1 to 0.7 moll, preferably between 0.2 and 0.6 moil of aqueous solution.

The thermal hydrolysis may take place in a sealed reactor at a temperature of between 110° C. and 200° C., preferably between 110° C. and 150° C., at autogenous pressure. The overpressure may reach a value of between 1 bar and 5 bar. The duration of the thermal hydrolysis treatment may be between 1 hour and 100 hours, preferably between 5 hours and 10 hours.

An ytterbium salt, and optionally an yttrium salt are added to the solution obtained, in amounts suitable for obtaining the desired stabilized zirconia powder.

The pH of the suspension is then rendered basic by addition of a base, in particular by addition of sodium hydroxide, potassium hydroxide or ammonium hydroxide, making it possible to ensure the precipitation of an ytterbium compound and optionally of an yttrium compound, in particular ytterbium hydroxides and optionally yttrium hydroxides, at the surface of the zirconia particles.

The suspension is then washed and rinsed, by any technique known to those skilled in the art, so as to recover particles.

The particles are then dried and subsequently calcined at a temperature between 950° C. and 1200° C. the whole time at this temperature preferably being between 1 hour and 5 hours.

The calcination makes it possible, as is well known, to modify the specific surface area of the powder of particles and also the mean size of the stabilized zirconia crystallites. In order to reduce the specific surface area of the powder, the calcination temperature and/or the hold time at the calcination temperature may be increased. To increase the mean size of the stabilized zirconia crystallites of the powder, the calcination temperature and/or the hold time at the calcination temperature may be increased.

Optionally, alumina and/or an alumina precursor are added to the powder of calcined particles.

The powder is then ground, for example using a ball mill according to any known technique, so as to obtain a zirconia powder having the desired median size.

The zirconia powder according to the invention may also be obtained by co-precipitation.

Preferably, no additive capable of modifying the prostitute, in particular the muco Prost to of the particles of the powder according to the invention is used in the process for manufacturing said powder.

Intermediate Product

The zirconia powder according to the invention is preferably made into an intermediate form suitable for its intended use.

The zirconia powder according to the invention may for example be made into the form of a feed powder, referred to as feedstock, more particularly intended for shaping by injection, into the form of a printing paste more particularly intended for shaping by 3D printing or into the form of a powder of granules more particularly intended for shaping by pressing.

Any conventional process can be used for making the zirconia powder according to the invention into an intermediate form.

The zirconia powder according to the invention, or the intermediate product resulting from the intermediate forming thereof (feedstock, printing paste, granules, etc.), is preferably packaged, for example in bags, pots, barrels or buckets, in order to be ready-to-use.

Process for Manufacturing a Dental Article According to the Invention

The manufacture of a dental article according to the invention comprises the steps a) and b) described above.

In step a), a feedstock suitable for the manufacture of a dental article is prepared.

The feedstock comprises a zirconia powder according to the invention, optionally in form of an intermediate product according to the invention, and optional constituents.

The amount of the optional constituents is preferably greater than 0.1% and/or less than 70%, as a weight percentage based on the weight of the dry feedstock, the zirconia powder according to the invention and/or the intermediate product according to the invention constituting the balance to 100% of the dry feedstock.

Depending on the process used for the shaping, a solvent, preferably water, may be added to the feedstock.

The optional constituents are the constituents conventionally used for the manufacture of sintered ceramic products. They include in particular the organic constituents and the coloring constituents.

In one embodiment, in particular when the shaping process in step b) is a slip casting process, the organic constituents are preferably chosen from dispersants, viscosity agents, antifoaming agents, and mixtures thereof, in an amount preferably greater than 0.1% and less than 3%, as a weight percentage based on the weight of the dry feedstock.

In one embodiment, in particular when the shaping process in step b) is a pressing process, the organic constituents are preferably chosen from binders, lubricants, resins and plasticizers, in an amount preferably greater than 0.5% and less than 10%, as a weight percentage based on the weight of the dry feedstock.

In one embodiment, in particular when the shaping process in step b) is a plastic injection process, the organic constituents are preferably chosen from surfactants, waxes, polymers, resins, plasticizers and mixtures, in an amount preferably greater than 25% and less than 65%, as a weight percentage based on the weight of the dry feedstock.

The coloring constituents are conventionally intended to give the dental article a color similar to that of teeth. They include in particular powders of known coloring pigments, in particular the oxides Er₂O₃, Fe₂O₃, MnO, and/or powders of zirconia, preferably that is at least partially stabilized, containing coloring pigments, optionally in the form of an intermediate zirconia product. The amount and the nature of the coloring constituents are adapted to the desired color.

In one embodiment, no coloring constituent is added to the feedstock.

In one embodiment, in particular when the powder according to the invention is in the form of an intermediate product according to the invention, no organic constituent is added to the feedstock.

The shaping of the feedstock comprising a zirconia powder according to the invention, optionally in the form of an intermediate product, may be carried out conventionally, by any technique known to those skilled in the art, in particular by slip casting, by pressing, in particular a uniaxial pressing or a cold isostatic pressing, by injection, in particular by plastic injection or by printing, in particular by 3D printing.

Preferably, the pressure applied during the uniaxial pressing is greater than 50 MPa and preferably less than or equal to 150 MPa.

A dental preform is thus obtained.

In one embodiment, step a) comprises the preparation of a blank, that is to say a slightly bulkier block than a tooth, ready to be machined in order to acquire the desired shape.

For this purpose, the zirconia powder according to the invention, optionally form of an intermediate product, is firstly formed into a “green” preform.

The green preform may have the shape of the blank or another shape, for example the shape of a disk.

The green preform may then undergo a heat treatment at a temperature above 800° C., preferably above 900° C. and below 1200° C., preferably below 1150° C. Said heat treatment preferably takes place in air, at atmospheric pressure. The whole time at the temperature plateau is preferably between 1 hour and 5 hours, preferably between 1 hour and 3 hours. Preferably, the green preform does undergo said heat treatment.

After the heat treatment, the green preform may also be cut and/or machined to obtain said blocks.

Each said heat-treated block constitutes a blank that can advantageously be handled.

The blank may be machined, preferably in a computer-assisted manner, in order to have the shape desired for the dental preform. The limited heat treatment advantageously enables it to remain easily machinable.

In one embodiment, step a) comprises the preparation of the dental preform by shaping the zirconia powder according to the invention, optionally into the form of an intermediate product, without producing a blank.

In step b), the dental preform is sintered at a temperature above 1400° C., preferably between 1400° C. and 1600° C., so as to obtain a dental article.

Preferably, said sintering is carried out at a temperature above 1420° C., preferably above 1450° C., and preferably below 1550° C., preferably below 1530° C. Said sintering preferably takes place in air, at atmospheric pressure. The hold time at the temperature plateau is preferably between 1 hour and 5 hours, preferably between 1 hour and 3 hours.

Preferably the rate of temperature rise up to the sintering plateau, preferably at least from at least from 400° C., is less than 1000° C./h, preferably less than 900° C./h, preferably less than 800′C. 1 h, preferably less than 700° C./h, preferably less than 600° C./h, or less than 400° C., 1 h, or less than 300° C./h, or less than 200° C./h, or less than 150° C./h, and, or greater than 30° C./h,

Preferably, the sintering takes place without application of an external pressure.

In one embodiment, the dental article is assembled with other articles in order to be used.

The dental article according to the invention may constitute an inlay core, an onlay, a dental implant, an artificial tooth, an orthodontic band, or a portion of said inlay core, artificial tooth or orthodontic band.

Sintered Dental Article

A dental article according to the invention is manufactured by means of a manufacturing process according to the invention.

Surprisingly, and without being able to explain it theoretically, the inventors have discovered that the presence of at least 1% Yb₂O₃ in the dental article, as a molar percentage based on the total molar content of ZrO₂, HfO₂, Yb₂O₃ and Y₂O₃, leads to a remarkable increase in its resistance to hydrothermal aging.

Preferably, the content of Yb₂O₃ is greater than or equal to 1.2%, preferably greater than or equal to 1.5% and/or less than or equal to 6.5%, preferably less than or equal to 6%, preferably less than or equal to 5.5%, preferably less than or equal to 5.2%, preferably less than or equal to 5%, preferably less than or equal to 4.7%, preferably less than or equal to 4.2%, preferably less than or equal to 4%, preferably less than or equal to 3.5%, preferably less than or equal to 3.2%, as molar percentages based on the sum of ZrO₂, HfO₂, Yb₂O₃ and Y₂O₃.

Preferably, the total content of Yb₂O₃+Y₂O₃ is greater than or equal to 2%, preferably greater than or equal to 2.5%, preferably greater than or equal to 3% and/or less than or equal to 6.5%, preferably less than or equal to 6%, preferably less than or equal to 5.5%, preferably less than or equal to 5%, as molar percentages based on the sum of ZrO₂, HfO₂, Yb₂O₃ and Y₂O₃.

Surprisingly, the inventors have also discovered that with low contents of Yb₂O₃+Y₂O₃, preferably of less than 5%, as a molar percentage based on the sum of ZrO₂, Yb₂O₃ and Y₂O₃, the dentai article simultaneously has a transmittance suitable for a dental application, a particularly high resistance to hydrothermal aging and a very good mechanical strength.

In one embodiment, the content of Y₂O₃ is less than 0.5%, less than 0.3%, or substantially zero, as molar percentages based on the sum of ZrO₂, HfO₂, Yb₂O₃ and Y₂O₃.

In one preferred embodiment, the content of Y₂O₃ is greater than or equal to 0.5%, preferably greater than or equal to 1%, preferably greater than or equal to 1.5% and less than 2%, preferably less than 1.9%, preferably less than 1.8%, as molar percentages based on the sum of ZrO₂, HfO₂, Yb₂O₃ and Y₂O₃.

Preferably, the content of Yb₂O₃ is greater than or equal to the content of Y₂O₃.

In a dental article, the content of Yb₂O₃+Y₂O₃ is adjusted so that the zirconia is at least partially stabilized, preferably completely stabilized, in the tetragonal crystallographic form, or in the tetragonal and cubic crystallographic forms.

The lower the fraction of monoclinic zirconia, the better the mechanical properties of the dental article.

Preferably, the fraction of monoclinic zirconia is less than 10%, preferably less than 5%, preferably less than 1%, preferably substantially zero.

In one embodiment, the content of Al₂O₃ is less than 0.01%, less than 0.006%, less than 0.005%, less than 0.003%, less than 0.002%, or substantially zero, as weight percentages based on the oxides,

Tests have however shown that alumina improves the mechanical strength of the dental article.

In one preferred embodiment, the content of Al₂O₃ is greater than or equal to 0.02%, preferably greater than or equal to 0.03%, preferably greater than or equal to 0.04% and less than or equal to 0.15%, preferably less than or equal to 0.10%, preferably less than or equal to 0.08%, as weight percentages based on the oxides.

The content of oxides other than ZrO₂, HfO₂, Yb₂O₃, Y₂O₃ and Al₂O₃ (“other oxides”) is as low as possible.

Preferably, the content of other oxides is less than 0.4%, preferably less than 0.3%, preferably less than 0.2%, preferably less than 0.1%, as weight percentages based on the oxides.

Preferably, the total content of oxide(s) of lithium, sodium, magnesium, silicon, cobalt, iron, manganese, chromium, nickel, copper, praseodymium, erbium, boron, potassium, calcium, titanium, niobium, barium, lanthanum, cerium, terbium and neodymium is less than 0.4%, preferably less than 0.3%, preferably less than 0.2%, preferably less than 0.1%, as weight percentages based on the oxides.

Preferably, the total content of cobalt oxides expressed in the form CoO, of iron oxides expressed in the form Fe₂O₃, of manganese oxides expressed in the form MnO, of chromium oxide expressed in the form Cr₂O₃, of nickel oxide NiO, of copper oxides expressed in the form CuO, of praseodymium oxide, of erbium oxide and of oxides of several of these elements is less than 0.05%, preferably less than 0.02%, preferably less than 0.01%, preferably less than 0.007%, preferably less than 0.005%, preferably less than 0.003%.

Preferably, the dental article according to the invention has a content of SiO₂ of less than 0.4%, preferably less than 0.2%, preferably less than 0.1%, preferably less than 0.05%, preferably substantially zero, as a weight percentage based on the weight of the dental article.

Preferably, the dental article according to the invention has a content of B₂O₃ of less than 0.05%, preferably less than 0.03%, preferably less than 0.02%, preferably less than 0.01%, preferably substantially zero, as a weight percentage based on the weight of the dental article. Boron may in fact potentially have a harmful effect on health.

Preferably, the dental article has a content of oxides of greater than or equal to 99%, preferably greater than or equal to 99.3%, preferably greater than or equal to 99.5%, preferably greater than or equal to 99.7%, preferably greater than or equal to 99.9%, as a weight percentage based on the weight of the sintered dental article.

A transmittance of greater than 34%, preferably greater than 35%, preferably greater than 36%, preferably greater than 37%, may be obtained by carrying out a process according to the invention, and in particular from the use of a zirconia powder according to the invention and a sintering at a temperature of between 1400° C. and 1600° C.

By using the preferred characteristics of the zirconia powder according to the invention and of the manufacturing process according to the invention, the dental article may advantageously have a transmittance of greater than or equal to 35%, preferably greater than 36%, preferably greater than 37% and/or less than 60%, preferably less than 50%, preferably less than 48%, preferably less than 45%, preferably less than 43%.

Remarkably, a dental article according to the invention also has a high radiopacity to x-rays.

Preferably, the dental article has a relative density of greater than 99.5%, preferably greater than 99.6%, preferably greater than 99.7%, preferably greater than 99.8%, preferably greater than 99.9%, the absolute density being calculated according to the method described previously.

Preferably, the dental article has a bulk density of greater than 6.15 g/cm³, preferably greater than 6.16 g/cm³ and/or preferably less than 6.51 g/cm³, preferably less than 6.45 g/cm³, preferably less than 6.36 g/cm³, preferably less than 6.30 g/cm³.

Preferably, the grains of the dental article have a mean size of less than 2 μm, preferably less than 1 μm, preferably less than 0.9 μm, preferably less than 0.8 μm, preferably less than 0.7 μm, and preferably greater than 0.1 μm, preferably greater than 0.2 μm.

Preferably, the dental article has a three-point bending strength of greater than 800 MPa, preferably greater than 900 MPa, or greater than 1000 MPa and less than 1500 MPa.

In one preferred embodiment, the dental article has:
a chemical analysis such that, as weight percentage based on the oxides:
   ZrO₂+HfO₂, which is (are) at least partially stabilized with Yb₂O₃, Y₂O₃ and mixtures thereof: balance to 100%, with HfO₂<2%,
   Yb₂O₃ and Y₂O₃ being present in an amounts such that, as molar percentages based on the sum of ZrO₂, HfO₂, Yb₂O₃ and Y₂O₃,
      3%≤Yb₂O₃+Y₂O₃≤5%, and
      Yb₂O₃≤1.2%, and
      Y₂O₃≤1.8%,
   0.03%≤Al₂O₃≤0.08%, and
   oxides other than ZrO₂, HfO₂, Yb₂O₃, Y₂O₃ and Al₂O: <0.2%, and
a transmittance, measured as described above, of greater than or equal to 34%.

Preferably in this embodiment, the dental article has a relative density of greater than 99.5%, preferably greater than 99.6%, preferably greater than 99.7%, preferably greater than 99.8%, preferably greater than 99.9%.

Treatment Process

A treatment process according to the invention comprises the following steps:

A) manufacturing a dental article according to the invention;

B) introducing the dental article into the mouth of an animal or a person, and preferably fastening the dental article in said mouth.

The treatment may be carried out for therapeutic purposes, for example consisting of an orthodontic treatment, or for nontherapeutic purposes, for example consisting of a purely esthetic treatment.

In step B), the dental article is preferably fastened, for example glued or screwed, onto a dental arch of said person or said animal.

In one embodiment, the dental article is fastened so as to be visibie, from the outside of the mouth of the animal or the person, when the person smiles and/or when the mouth of the animal or person is opened,

Preferably, the dental article is then kept in position for a period of more than a week, 2 weeks, 10 weeks or 40 weeks.

### EXAMPLES

The following nonlimiting examples are given for the purpose of illustrating the invention.

Measurement Protocols

The following methods were used to determine certain properties of the zirconia powders and of the sintered products obtained from said powders. They allow an excellent simulation of the actual behavior in service in a dental application.

The bulk density of the sintered products is measured by hydrostatic weighing.

The lattice parameters necessary for the calculation of the absolute density of the at least partially stabilized zirconia are determined by x-ray diffraction and the surface of the sample to be characterized (the sample not having been ground in the form of a powder) by means of a D8 Endeavor machine from the company Bruker. The parameters necessary for the acquisition of the diffraction pattern are identical to those used for the acquisition of the diffraction pattern necessary for determining the fraction of monoclinic zirconia.

The lattice parameters a and c are determined after having carried out a refinement of the diffraction pattern using the Fullprof software available on the site https://www.ill.eu/sites/fullprof/, using a pseudo-voigt profile (with npr=5), the refined parameters being the following:
the sample displacement using the “SyCos” function,
the Lorentzian/Gaussian proportion of the peudo-voigt function using the “Shape 1” function,
the full width at half maximum parameters U, V, W,
the asymmetry parameters using the “Acy1” and “Asy2” functions,
the points of the baseline,
   the space group of the partially substituted tetragonal zirconia lattice being P 42/n m c (137), which is considered identical to that of the unsubstituted tetragonal zirconia lattice.

The chemical analysis of the sintered products is measured by Inductively Coupled Plasma, or ICP, for the elements with a content that does not exceed 0.5%. In order to determine the content of the other elements, a bead of the product to be analyzed is manufactured by melting the product, then the chemical analysis is carried out by x-ray fluorescence.

The mean grain size of the sintered products is measured by the “Mean Linear Intercept” method. A method of this type is described in the standard ASTM E1382.

According to the standard, analysis lines are plotted on images of the sintered products, then, along each analysis line, “intercept” lengths are measured, between two consecutive grain boundaries intersecting said analysis line.

The mean length “I” of the intercepts “I” is then determined.

For the tests below, the intercepts were measured on images, obtained by scanning electron microscopy, of samples of sintered products, said sections having previously been polished until a mirror quality was obtained then thermally attacked, at a temperature 50° C. lower than the sintering temperature, to reveal the grain boundaries.

The magnification used for taking the images is chosen so as to see around 100 grains on the one image. 5 images per sintered product were produced.

The mean grain size “d” of a sintered product is given by the relation: d−1.56.I′. This formula is derived from the formula (13) from the article “Average Grain Size in Polycrystalline Ceramics”, M. I. Mendelson, J. Am. Ceram. Soc., Vol. 52, No. 8, pp. 443-446.

The specific surface area of a powder is measured by the BET (Brunauer Emmet Teller) method described in the Journal of the American Chemical Society, 60 (1938), pages 309 to 316.

The median size of a powder is measured conventionally using a LA950V2 model laser particle size analyser sold by the company Horiba.

The mean size of the crystallites of stabilized zirconia, D, of a zirconia powder is determined by x-ray diffraction on the surface of the sample to be characterized (the sample not being ground in the form of a powder) by means of a D8 Endeavor machine from the company Bruker, using the following equation:

$$D = \frac{K λ}{\sqrt{( {B}^{2} - {b}^{2} )} cos θ} \times \frac{1}{10} \times \frac{180}{π}$$

K being equal to 0.89, λ being the wavelength of the x-rays, here equal to 1.5418 angstroms, B being the full width at half maximum of the peak of the (111) plane of the stabilized zirconia, in degrees, b being the full width at half maximum of the peak of the single-crystal silicon standard used, and 2θ being the angle of the maximum intensity of the peak corresponding to the (111) plane of the stabilized zirconia, in degrees.

The acquisition of the diffraction patterns of the single-crystal silicon standard and of the example is carried out over an angular range 2θ of between 5° and 100°, with a step of 0.01°, and a count time of 0.34 s/step. The front lens comprises a primary slit of 0.3° and a Soller slit of 2.5°. The sample characterized is rotated about itself at a speed equal to 15 rpm, with use of the automatic knife. The rear lens comprises a Soller slit of 2.5°, a nickel filter of 0.0125 mm and a 1D detector with an aperture equal to 4°.

After having eliminated the Kα2 line, the full width at half maximum of the peaks is determined using the HighScore Plus software, The deconvolution function used is an asymmetric split-width pseudo-Voigt function. The standard and the samples are deconvoluted under the same conditions.

The transmittance (in %) of the sintered products of the examples is measured at a wavelength of the incident radiation and of the transmitted radiation equal to 600 nm at ambient temperature (20° C.) on samples in the form of plates with a thickness equal to 1 mm having a roughness Ra<10 nm, using an X-Rite, Color i5 Benchtop model spectrophotometer, comprising a xenon lamp emitting instant light calibrated on the D65 standard illuminant and having a wavelength range between 360 nm and 750 nm.

The 3-point bending strength is determined on bars of sintered product having the dimensions 25×6.7×3 mm³, said bars being on supports spaced 20 mm apart and centred with respect to the length of said bar during the bending test, the load being applied with a rate of displacement equal to 0.5 mm/min,

The resistance to hydrothermal aging of the sintered products of the examples is evaluated by the following method.

Each sample, having the shape of the disk with a diameter equal to 25 mm and thickness equal to 2 mm, is polished on one of the main faces using disk of sandpaper having an abrasive particle size equal to 3 μm. The polishing is carried out so as not to generate monoclinic zirconia on the polished surface.

The polish samples are then subjected to an accelerated ageing test according to the following protocol: the samples are placed in a Teflon crucible with a diameter equal to 80 mm and a capacity equal to 0.5 liter. Said crucible is placed in an autoclave with a diameter equal to 100 mm and a capacity equal to 1 liter. 100 ml of water are added to the autoclave, outside of the crucible. The autoclave is sealed and everything is brought to a temperature of 135° C. for 5 hours, at autogenous pressure.

Before testing, all the sintered products of the examples are completely stabilized. The measurement of the fraction of monoclinic zirconia, as described above, on samples that have undergone the treatment in an autoclave, therefore directly provides a measurement of the resistance to hydrothermal aging.

Manufacturing Protocol

The sintered product of the comparative example 1 was obtained from zirconia powder in the form of granules with a median size equal to 48 μm, and having, after thermal debinding in air at 500° C. for 3 hours, the characteristics that appear in table 1.

The sintered product of the comparative example 2 was obtained from zirconia powder in the form of granules with a median size equal to 52 μm, and having, after thermal debinding in air at 500° C. for 3 hours, the characteristics that appear in table 1,

The zirconia powder of example 3, according to the invention, that made it possible to obtain the sintered product of example 3, was manufactured by the following process.

A zirconium oxychloride is added to demineralized water, in an amount such that the molar concentration of zirconium is equal to 0.5 mol/l. Added next are a Yb₂O₃ powder haying a weight purity of greater than 99.99% and having a median size equal to 5.8 μm and a Y₂O₃ powder having a weight purity of greater than 99.999% and having a median size equal to 6.6 μm, in a content equal to 1.85 mol % and 1.78 mol %, respectively, based on the content of Yb₂O₃, of Y₂O₃ and of zirconia equivalent provided by the zirconium oxychloride, Next, a thermal hydrolysis is carried out in a sealed reactor at a temperature equal to 140° C., at autogenous pressure (the overpressure being substantially equal to 3 bar). The duration of the thermal hydrolysis treatment is equal to 6 hours. The pH of the suspension obtained is brought to a value of greater than 9 by the addition of an aqueous 20 wt% ammonium hydroxide solution. The suspension obtained is then filtered using a filter press and rinsed with demineralized water,

The particles are then dried at 600° C. for 3 hours. Next, the particles are calcined in air at 1080° C., the hold time at this temperature being equal to 3 hours. After calcination, an alumina powder having a weight purity of greater than 99.99% and having a median size equal to 0.2 μm is added to the calcined powder, then everything is ground using a ball mill.

The characteristics of the zirconia powder of example 3 appear in table 1.

TABLE 1

| | Example | Example | Example |
|---|---|---|---|
| Powder | 1(*) | 2(*) | 3 |

Chemical analysis as weight percentages based on the oxides

| ZrO₂ + HfO₂ + Yb₂O₃ + Y₂O₃ | Balance to 100% | | |
|---|---|---|---|
| Al₂O₃ | 0.05 | 0.05 | 0.04 |
| oxides other than ZrO₂, HfO₂, | <0.05 | <0.05 | <0.05 |
| Yb₂O₃, Y₂O₃ and Al₂O₃ | | | |

Chemical analysis as molar percentages based on ZrO₂ + HfO₂ +
Yb₂O₃ + Y₂O₃

| | | | |
|---|---|---|---|
| Yb₂O₃ | 0 | 0 | 1.85 |
| Y₂O₃ | 3.10 | 4.10 | 1.78 |
| Yb₂O₃ + Y₂O₃ | 3.10 | 4.10 | 3.63 |

Other characteristics of the powder

| | | | |
|---|---|---|---|
| Specific surface area (m²/g) | 11.5 | 9.0 | 9.4 |
| Median size D₅₀ (μm) | 0.13 | 0.14 | 0.15 |
| Mean size of crystallites of | 32 | 36 | 42 |
| stabilized zirconia (in nm) | | | |

(*)example outside the invention

Granules of the zirconia powder according to example 3 were then produced by spray drying of a suspension containing 50% by weight of water, 50% by weight of the powder according to example 3, the suspension also containing 3% of polyvinyl alcohol and 1% of polyethylene glycol 3000, the percentages of polyvinyl alcohol and of PEG3000 being weight percentages based on the weight of the zirconia powder according to example 3. The powder of granules obtained has a median size equal to 56 μm and an untapped weight per liter equal to 1.4 g/cm³,

Each of the examples, the powder of granules is shaped by uniaxial pressing at a pressure equal to 100 MPa, in the form of disks having a diameter equal to 32 mm and thickness equal to 2.5 mm, with the exception of the bars needed for the measurements of modulus of rupture, which have a parallelepipedal shape, of which the length is equal to 32 mm, the width equal to 8 mm and the thickness equal to 4 mm.

The preforms obtained are then transferred into a sintering furnace where they are brought, at a rate of 100° C./h, up to 400° C., held at this temperature for 2 hours, then brought at a rate of 100° C./h to 1530° C., and maintained at this temperature for 2 hours,

The decrease in temperature is carried out by natural cooling,

The sintering leads to a shrinkage of the samples,

Results

The results obtained for the sintered products manufactured from the zirconia powders from examples 1 to 3 are summarized in table 2 below.

TABLE 2

| | Example | Example | Example |
|---|---|---|---|
| Sintered product | 1(*) | 2(*) | 3 |

Chemical analysis as weight percentages based on the oxides

| ZrO₂ + HfO₂ + Yb₂O₃ + Y₂O₃ | Balance to 100% | | |
|---|---|---|---|
| Al₂O₃ | 0.05 | 0.05 | 0.04 |
| oxides other than ZrO₂, HfO₂, | <0.05 | <0.05 | <0.05 |
| Yb₂O₃, Y₂O₃ and Al₂O₃ | | | |

Chemical analysis as molar percentages based on ZrO₂ + HfO₂ +
Yb₂O₃ + Y₂O₃

| | | | |
|---|---|---|---|
| Yb₂O₃ | 0 | 0 | 1.85 |
| Y₂O₃ | 3.10 | 4.10 | 1.78 |
| Yb₂O₃ + Y₂O₃ | 3.10 | 4.10 | 3.63 |

Other characteristics of the sintered product

| | | | |
|---|---|---|---|
| Transmittance (%) | 37.8 | 38.7 | 38.7 |
| Bulk density (g/cm³) | 6.08 | 6.06 | 6.22 |
| Relative density (%) | n.d. | n.d. | >99.9 |
| Mean grain size (μm) | 0.43 | 0.56 | 0.57 |
| Fraction of monoclinic zirconia (%) | <5 | <5 | <5 |
| 3-point bending strength (MPa) | 1060 | 1025 | 1020 |
| Resistance to hydrothermal aging | 35 | 20 | 14 |
| (traction of monoclinic zirconia | | | |
| after test, in %) | | | |

(*)example outside the invention
n.d.: not determined

The sintered products of examples 1 to 3 have a total content of cobalt oxides expressed in the form CoO, of iron oxides expressed in the form Fe₂O₃, of manganese oxides expressed in the form MnO, of chromium oxide expressed in the form Cr₂O₃, of nickel oxide NiO, of copper oxides expressed in the form CuO, of praseodymium oxide, of erbium oxide and of oxides of several of these elements of less than 0.005%.

A comparison of the sintered products shows that the sintered product according to example 3 has, after the hydrothermal aging test, a fraction of monoclinic zirconia equal to 14%, that is to say 2,5 times lower than that of the sintered product according to example 1, equal to 35%, and 1.4 lower than that of the sintered product according to example 2, equal to 20%; all these products, having undergone the same sintering heat treatment and having a substantially identical alumina content, being advantageously comparable.

Surprisingly, it is observed that the fraction of monoclinic zirconia after the hydrothermal aging test of example 3 according to the invention, equal to 14%, is lower than that of the comparative example 2, equal to 20%, even though example 2 comprises more stabilizer, The inventors attribute this unexpected result in the presence of ytterbium oxide according to the invention.

As is now clearly apparent, the invention thus makes it possible to manufacture a dental article having a high resistance to hydrothermal aging, perfectly suitable for a dental application.

Of course, the invention is not limited to the examples and embodiments described above.

1. A powder intended for the manufacture of a sintered dental article, the powder having
a chemical analysis such that, as weight percentages based on the oxides:
Al₂O₃: ≤0.2%,
oxides other than ZrO₂, HfO₂, Yb₂O₃, Y₂O₃ and Al₂O₃: <0.5%,
ZrO₂+HfO₂+Yb₂O₃+Y₂O₃: balance to 100%, with HfO₂<2%, the contents of Yb₂O₃ and Y₂O₃, as molar percentages based on the sum of ZrO₂, HfO₂, Yb₂O₃ and Y₂O₃, being such that Yb₂O₃≥1%, 0.5%≤Y₂O₃<2%, and Yb₂O₃+Y₂O₃≤5.5%,
a specific surface area of greater han or equal to 5 m²/g and less than or equal to 16 m²/g, and
a median size of greater than or equal to 0.1 μm and less than or equal to 0.7 μm.

2. The powder as claimed in claim 1, wherein said content of Yb₂O₃ is greater than or equal to 1.2%.

3. The powder as claimed in claim 1, wherein said content of Yb₂O₃ is less than or equal to 3.5%.

4. The powder as claimed in claim 1, wherein said content of Y₂O₃ is less than 1.8%.

5. The powder as claimed in claim 4, wherein said content of Y₂O₃ is greater than or equal to 1%.

6. The powder as claimed in claim 1, wherein the sum of said contents of Yb₂O₃ and of Y₂O₃ is greater than or equal to 3% and/or less than or equal to 5%.

7. The powder as claimed in claim 1, wherein said content of Yb₂O₃ is greater than or equal to said content of Y₂O₃.

8. The powder as claimed in claim 1, wherein Al₂O₃≥0.03%, as weight percentages based on the oxides.

9. The powder as claimed in claim 1, wherein Al₂O₃≤0.10%, as weight percentages based on the oxides.

10. The powder as claimed in claim 1, wherein
said contents of Yb₂O₃ and Y₂O₃, as molar percentages based on the sum of ZrO₂, HfO₂, Yb₂O₃ and Y₂O₃, are such that 3% ≤Yb₂O₃+Y₂O₃≤5%, Yb₂O₃≥1.2% and Y₂O₃≤1.8%,
the content of Al₂O₃, as weight percentage based on the oxides, is such that 0.03%≤Al₂O₃≤0.08%,
the content of oxides other than ZrO₂, HfO₂, Yb₂O₃, Y₂O₃ and Al₂O₃, as weight percentage based on the oxides, is less than 0.2%,
the specific surface area is greater than or equal to 8 m²/g and less than or equal to 12 m²/g, and
the median size is greater than or equal to 0.1 μm and less than or equal to 0.3 μm.

11. An intermediate product consisting of particles bound by means of an organic product, said particles together forming, after debinding of the intermediate product, a powder as claimed in claim 1.

12. A process manufacturing a dental article, said process comprising the following steps:
a) preparing and shaping a feedstock comprising a powder as claimed in claim 1, optionally in the form of an intermediate product as claimed in claim 11, so as to obtain a dental preform;
b) sintering said dental preform at a temperature above or equal to 1400° C., so as to obtain a dental article.

13. A sintered dental article having a chemical analysis such that, as eight percentages based on the oxides:
Al₂O₃: ≤0.2%,
oxides other than ZrO₂, HfO₂, Yb₂O₃, Y₂O₃ and Al₂O₃: <0.5%,
ZrO₂+HfO₂+Yb₂O₃+Y₂O₃: balance to 100%. with HfO₂<2%,
the contents of Yb₂O₃ and Y₂O₃, as molar percentages based on the sum of ZrO₂, HfO₂, Yb₂O₃ and Y₂O₃, being such that Yb₂O₃≥1%, 0.5%≤Y₂O₃<2%, and Yb₂O₃+Y₂O₃≤5.5%,
ZrO₂ being at least partly stabilized with Yb₂O₃, and optionally with Y₂O₃,
the dental article having a transmittance of greater than or equal to 34%, the transmittance being the Iₜ/Iᵢ ratio, wherein
Iᵢ denotes the intensity of the radiation with a wavelength of 600 nm of light calibrated on the D65 standard illuminant and projected, at a temperature of 20° C., on a plate extracted from said dental article and having a thickness equal to 1 mm, and,
Iₜ denotes the intensity of the radiation with a wavelength of 600 nm of said light after it has passed through said plate,
the surfaces passed through by said radiation having a roughness Ra of less than 10 nm.

14. The dental article as claimed in the preceding claim 13, wherein:
1.2%≤Yb₂O₃≤4.2%, as molar percentages based on the sum of ZrO₂, HfO₂, Yb₂O₃ and Y₂O₃, and/or
3%≤Yb₂O₃+Y₂O₃≤5%, as molar percentages based on the sum of ZrO₂, HfO₂, Yb₂O₃ and Y₂O₃, and/or
Y₂O₃<1.8%, as molar percentages based on the sum of ZrO₂, HfO₂, Yb₂O₃ and Y₂O₃, and/or
0.02%≤Al₂O₃≤0.10%, as weight percentages based on the oxides, and/or
the content of oxides other than ZrO₂, HfO₂, Yb₂O₃, Y₂O₃ and Al₂O₃ is less than 0.2%, as weight percentages based on the oxides, and/or
the transmittance is greater than 36%, and/or
the transmittance is less than 48%, and/or
the relative density is greater than 99.5%, and/or
the mean grain size is greater than 0.1 μm and less than 1 μm.

15. The dental article as claimed in claim 13, selected from the group consisting of an inlay core, an onlay, a dental implant, an artificial tooth, an orthodontic band, or a portion of said artificial tooth, orthodontic band or inlay core.
